(19) United States
(12) Patent Application Publication
(10) Pub. No.: US 2019/0054004 A1
(43) Pub. Date: Feb. 21, 2019

(54) EMULSION COMPRISING SILICONE FLUID COMPOSITION FOR HAIR CARE APPLICATION

(71) Applicant: WACKER METROARK CHEMICALS PVT. LTD., Chandi, West Bengal (IN)

(72) Inventors: Amit Kumar PAUL, Kolkata, West Bengal (IN); Ayndrila DUTTA, Kolkata, West Bengal (IN); Sunanda BISWAS, Dist: 24-Parganas (N), Kanchrapara, West (IN)

(73) Assignee: WACKER METROARK CHEMICALS PVT. LTD., Chandi, West Bengal (IN)

(21) Appl. No.: 15/759,142

(22) PCT Filed: Sep. 10, 2016
(86) PCT No.: PCT/IB2016/055410

§ 371 Date: Mar. 9, 2018

(30) Foreign Application Priority Data
Sep. 11, 2015 (IN) 973/KOL/2015

(51) Int. Cl. A61K 8/893; A61K 8/06; A61Q 5/02; A61Q 19/10; A61Q 19/00; A61Q 5/12

(52) U.S. Cl. CPC A61K 8/893; A61K 8/062; A61Q 5/02; A61K 2800/21; A61Q 19/00; A61Q 5/12; A61Q 19/10

(57) ABSTRACT

The present invention relates to a silicone fluid comprising a glycoside group and oil in water emulsions composition of silicone fluid used in hair care compositions for hair care applications, process for their preparations and their uses. The current invention is to provide an oil-in-water emulsion used as a shampoo additive composition that will help hair care application for shine as well as easy manageability and alignment along with conditioning benefits. The emulsion having a D₅₀ particle size less than 3 micrometers comprising a silicone fluid, a mixture of non-ionic emulsifier; and water, wherein, the silicone fluid is of viscosity of at least 4500 mPa·s at 25° C.

### FIELD OF INVENTION

The present invention relates to oil in water emulsion compositions of silicone fluid comprising a glycoside group for hair care compositions preferably for hair care applications, process for their preparations and their uses.

### BACKGROUND

Today in the busy schedule and fast life today's consumer do not have time for spending on hair styling. Today's consumer needs quick and easy solutions that give the desired effect which is long lasting. Usually in day to day hair care the consumers along with cleaning and conditioning effect desire the effect of shine and manageability.

There are documents related to hair application. US20140017185A1 describes the a conditioner in the form of an optically non-transparent dispersion containing at least one cationic surfactant, a micro emulsion containing b1) at least one alkyl glycoside and/or an alkyl oligoglycoside, b2) at least one co-surfactant which does not fall under the definition of b1), b3) an organic oil phase, and b4) water, at least one fatty alcohol, optionally further surfactants and optionally further cosmetic additives, wherein the sum of all the surfactants present in the conditioner makes up a proportion of at most 10 wt % of the conditioner. This document also relates to the use and production thereof.

DE19719121C1 relates to novel hair treatment agents, containing (a) nacreous waxes and (b) glycosidic silicone compounds of formula (A) R²ₓR¹₃₋ₓSiO—[(SiR¹R²O)ₘ—(SiR¹₂O)ₙ]_{y}—SiR¹₃₋ₓR²ₓ, wherein R¹ represents hydrogen or an optionally substituted alkyl and/or alkenyl radical with 1 to 18 carbon atoms, R² represents a radical of formula (B) R³(R⁴O)_{c}[G]ₚ, wherein R³ represents alkylene radicals with 1 to 18 carbon atoms, R⁴ represents alkylene radicals with 2 to 4 carbon atoms, G represents glycoside radicals with 5 to 12 carbon atoms and p represents numbers from 1 to 10, c represents 0 or numbers from 1 to 20, m represents 0 or numbers from 1 to 200, n represents 0 or numbers from 1 to 1000, x represents 0 or 1 and y represents 0 or numbers from 1 to 1200, on the condition that (I) contains at least one radical R². The inventive agents of this document have a nacreous shine, make hair pleasant to touch and show no tendency towards phase separation, regardless of the particle size of the nacreous crystals.

EP1093844 (A1) describes Emulsifying system comprising (1) at least one alkyl polyglycoside with HLB (hydrophilic/lipophilic balance) below 7 (preferably up to 5) and (2) at least one oxyalkylenated polydimethyl siloxane. System contains 15-60 wt. % (per active matter) of (1) and 40-85 wt. % (per active matter) of (2), and may also contain fatty alcohol containing alkyl group corresponding to alkyl radical of alkyl polyglycoside. Independent claims are also included for: (1) a water/oil emulsion for use as cosmetic composition comprising aqueous phase dispersed in oil phase (oil phase content 5-50 wt. % per total wt. of emulsion), emulsifying system as claimed, preferably in amount 0.5-20 wt. % per total wt. of emulsion, optional co- emulsifying system selected from alkyl esters of polyols, and optionally at least one filler; (2) use of emulsion as claimed in cosmetics, for treatment, protection, care, make-up removal and/or cleaning of skin, lips and/or hair, and/or for make-up of skin and/or lips; (3) a process for cosmetic treatment of skin, hair and/or lips, comprising application of emulsion as claimed; (4) use of the emulsion as claimed in preparation of cosmetic composition for treating dry and/or sensitive skin and dry lips; and (5) use of the emulsifying system as claimed in preparation of water/oil type emulsions.

JP2005336059A describes a hair cosmetic which exhibits enhancing effects of feelings such as smoothness or suppleness similarly to silicone or a cationized cellulose, hardly exhibits build-up properties and does not inhibit effects of a hair-dyeing agent or a permanent hair treatment agent, and a feeling-enhancing agent exhibiting above-mentioned effects. The hair cosmetic contains an alkyl glucoside bearing a 4-8C alkyl group and a glycol monoalkyl ether at a specific ratio.

U.S. Pat. No. 6,066,326 A describes the use of a polydimethylsiloxane containing glucoside groups as a moisturizing agent in a cosmetic or dermatological composition. The polydimethylsiloxane compound that contains glucoside groups is especially effective for treating human skin and the scalp, and is particularly effective for moisturizing the skin and treating dry skin.

US 20090041710A1 describe the shine of hair fiber that bears phenyl siloxy units.

There are prior arts that recite high refractive index compounds such as phenyl silicones with higher refractive index for shine benefits on the hair or keratin fibers. But such high refractive index compounds do not give long lasting shine benefit.

There are prior arts that describe alkyl polyglycoside in the personal care composition, but they are mainly used as emulsifier. Also there are few prior arts that mention poly dimethyl siloxane containing glycoside group as moisturizing agent and having emolliency properties, primarily in skin care application.

There is a need of a novel personal care, preferably hair care solution that imparts long lasting shine by aligning the hair strand and also giving better feel properties and manageability.

Thus one of the objectives of the current invention is to overcome the drawbacks of the prior art.

A further object is to provide a styling solution that will help the consumer for hair care application for shine as well as easy manageability and alignment along with conditioning benefits.

Another objective of the current invention is to provide stable oil in water emulsion that provides styling and conditioning benefits when added with a hair composition.

### SUMMARY OF THE INVENTION

An oil-in-water emulsion comprising:

a) a silicone fluid of formula (I)

R⁵₃SiO(R¹₂SiO)ₘ(R¹R²SiO)ₙ(YR¹SiO)ₚ—SiR⁵₃ (I)

where, Y is a radical of formula Z(R³O)_{f}R⁴—,
n and p are numbers from 0 to 10000 and m is a number from 200 to 10000,
R¹ is selected from a C₁ to C₆ alkyl group, R² is a C₁ to C₁₈ alkyl group,
R³ is a C₁ to C₆ alkylene radical,
R⁴ is a C₁ to C₆ alkylene radical,
R⁵ is selected from a C₁ to C₆ alkyl group, C₁ to C₆ alkoxy group, hydroxyl group,
Z is a glycoside radical of from 1 to 100 monosaccharide units or its derivatives;
b) a mixture of emulsifier comprising one or more non-ionic emulsifier; and
c) water.

In one of the embodiment, a personal care composition comprising: an emulsion comprising:

a) a silicone fluid of formula I of viscosity of at least 4500 mPa·s at 25° C.;
b) a mixture of non-ionic emulsifier; and
c) water.

In one of the other embodiment, a personal care composition comprising:

b) a silicone fluid of formula (I)

R⁵₃SiO(R¹₂SiO)ₘ(R¹R²SiO)ₙ(YR¹SiO)ₚ—SiR⁵₃ (I)

where, Y is a radical of formula Z(R³O)_{f}R⁴—,
n and p are numbers from 0 to 10000 and m is a number from 200 to 10000,
R¹ is selected from a C₁ to C₆ alkyl group, R² is a C₁ to C₁₈ alkyl group,
R³ is a C₁ to C₆ alkylene radical,
R⁴ is a C₁ to C₆ alkylene radical,
R⁵ is selected from a C₁ to C₆ alkyl group, C₁ to C₆ alkoxy group, hydroxyl group,
Z is a glycoside radical of from 1 to 100 monosaccharide units or its derivatives.

A further embodiment comprises method of preparing an silicone oil-in-water emulsion having a D₅₀ particle size less than 3 micrometers comprising:

rearranging caprylyl dimethicone ethoxy glucoside of viscosity from 40 to 100 mPa·s at 25° C. which is available as SPG128 VP from Wacker having INCI name cyclopentasiloxane, caprylyl dimethicone ethoxy glucoside with polydimethylsiloxane of viscosity from 40 to 100 mPa·s at 25° C. and trialkyl terminated polydialkylsiloxane of viscosity from 5 to 50 mPa·s at 25° C. at a temperature of 100 to 175° C. to obtain oil-in water emulsion of silicone fluid of formula I of viscosity of at least 4500 mPa·s at 25° C.

### DETAILED DESCRIPTION OF THE INVENTION

Hair alignment is the key for shine. To use higher refractive index silicone for improving shine is readily used in the hair care application e.g. phenyl silicone, but in reality, due to higher static electricity generation with such high refractive silicone material, hair is never aligned and no shine is observe. Without wish to be bound by theory, the present inventors have found that high viscosity silicone composition of the present invention on one hand make hydrophobic coating on hair surface and simultaneously discharge static electricity due to the presence of glycoside groups. Therefore, after using shampoo or conditioner having silicone glycoside, hair is perfectly aligned and long lasting shine is observed on hair due to low surface tension of silicone glycoside, consumer also feel faster clean feel due to faster removal of shampoo residues from hair during rinsing.

In the silicone fluid of formula (I) as mentioned above,

R⁵₃SiO(R¹₂SiO)ₘ(R¹R²SiO)ₙ(YR¹SiO)ₚ—SiR⁵₃ (I)

where, Y is a radical of formula Z(R³0)_{f}R⁴-, n and p are numbers from 0 to 10000 and m is a number from 200 to 10000, p is preferably greater than zero. In one of the embodiment, the R² group is preferably a C₂ to C₁₈ alkyl group in the formula I.

In another embodiment, the silicone fluid is of viscosity of at least 4500 mPa·s at 25° C., where the silicone fluid comprises a glycoside group.

The silicone fluid of formula I is prepared by hydrosilylation reaction by reacting monosaccharide with an organosilicon compound containing at least one Si-bonded hydrogen.

The silicone fluid is then emulsified with a mixture of non-ionic emulsifier and water to obtain the silicone oil-in water emulsion, wherein the silicone fluid comprises a glycoside group

Preferably the personal care composition of the present invention comprises a silicone fluid of viscosity of at least 4500 mPa·s at 25° C., more preferably, the personal care composition is in the form of an emulsion.

In a further embodiment, the personal care composition further comprises an emulsifier. In one of the embodiments, the emulsifier is a mixture of non-ionic emulsifier.

The personal care composition of the silicone fluid of formula (I) further comprises a cosmetic solvent which is selected from any solvent used in the personal care application and selected from a silicone or non-silicone fluid of personal care grade and preferably from a low viscosity silicone solvent, cyclic silicone, a hydrocarbon or its derivative of personal care grade more preferably a solvent from C₁₀-C₂₀ alkane solvent. Such personal care composition may in one of the examples be used for a hair leave-on application.

The silicone fluid of the present invention has viscosity of at least 4500 mPa·s at 25° C. In one of the embodiments, the silicone fluid having viscosity of from 4500 to 15000 mPa·s at 25° C. The silicone fluid may also have high viscosity from 15,000 to 100,000 mPa·s at 25° C. The caprylyl dimethicone ethoxy glucoside of viscosity from 40 to 100 mPa·s at 25° C. which is available as SPG128 VP is prepared by a process for the preparation of an organosilicon compound containing glycoside radicals, which comprises,

in a first stage reacting, in the absence of an organic solvent, a monosaccharide and/or oligosaccharide of the formula

HO—(R³O)—R⁴ (II)

in which R³ is an alkylene radical, and R⁴ is an alkenyl radical, in the presence of an acid, and
in a second stage neutralizing the acid and reacting the glycoside-containing compound obtained in the first stage with an organosilicon compound containing at least one Si-bonded hydrogen.

The glucoside molecules may be selected from a group but may not be restricted to monosaccharide, polysaccharide. The glucoside or glycoside may be interchangeably used in this application. A glycoside is a molecule in which a sugar is bound to another functional group via a glycosidic bond. A glucoside is a glycoside that is derived from glucose.

Examples of monosaccharides from which the glycoside radicals Z can be built up are hexoses and pentoses, such as glucose, fructose, galactose, mannose, talose, allose, altrose, idose, arabinose, xylose, lyxose and ribose, glucose being preferred. Z is a glycoside radical of from 1 to 100 monosaccharide units or its derivatives and preferably from 1 to 10 monosaccharide units or its derivatives.

Examples of alkylene radicals are methylene, ethylene, propylene, butylene, pentylene, hexylene, heptylene, octylene, nonylene, decylene and octadecylene radicals.

Examples of hydrocarbons R¹, R² in the formula I are alkyl radicals, such as the methyl, ethyl, n-propyl, isopropyl, 1-n-butyl, 2-n-butyl, isobutyl, tert-butyl, n-pentyl, isopentyl, neopentyl and tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radicals, decyl radicals, such as the n-decyl radical, dodecyl radicals, such as the n-dodecyl radical, and octadecyl radicals, such as the n-octadecyl radical; alkenyl radicals such as the vinyl and ally radical; cycloalkyl radicals, such as the cyclopentyl, cyclohexyl, cycloheptyl and methylcyclohexyl radicals; aryl radicals, such as the phenyl, naphthyl, anthryl and phenanthryl radical; alkaryl radicals, such as the o-, m- and p-tolyl radicals, xylyl radicals and ethylphenyl radicals; and aralkyl radicals such as the benzyl radical and the \tilde{□} and the □-phenylethyl radical or caprylyl radical. Most preferred R¹ is the methyl radical and R² is octyl or caprylyl radical.

The radicals R³ in formula I is preferably the ethylene radical and the 1,2-propylene radical, the ethylene radical being more preferred.

The radical R⁴ in formula I is preferably linear alkylene radicals having from 2 to 20 carbon atoms, more preferably linear alkylene radicals having from 2 to 8 carbon atoms, in particular the n-propylene radical.

Examples of radicals Y in formula I are Z—CH₂CH₂CH₂—, Z—(CH₂CH₂O)—CH₂CH₂CH₂—, Z—(CH₂CH₂O)₂—CH₂CH₂CH₂—, Z—(CH₂CH(CH₃)O)—CH₂CH₂CH₂—, Z—(CH₂CH(CH₃)O)₂—CH₂CH₂CH₂—, Z—(CH₂CH₂O)—CH₂CH₂CH(CH₃)CH₂—, Z—(CH₂CH₂O)₂—CH₂CH₂CH(CH₃)CH₂— in which Z represents a glycoside radical (C₆H₁₁O₆—); Z₂—CH₂CH₂CH₂—, Z₂—(CH₂CH₂O)—CH₂CH₂CH₂—, Z₂—(CH₂CH₂O)₂—CH₂CH₂CH₂—, Z₂—(CH₂CH(CH₃)O)—CH₂CH₂CH₂—, Z₂—(CH₂CH(CH₃)O)₂—CH₂CH₂CH₂—, Z₂—(CH₂CH₂O)—CH₂CH₂CH(CH₃)CH₂—, Z₂—(CH₂CH₂O)₂—CH₂CH₂CH(CH₃)CH₂—, where Z₂built up from two glucose units.

The silicone fluid comprising a glycoside unit preferably has the hydroxyl terminated or methoxy terminated silicone fluid or mixture of both hydroxyl and methoxy terminated silicone fluid is less than 1000 ppm. Silicone fluid has less than 1000 ppm by weight of hydroxyl terminated dialkylpolysiloxane or methoxy terminated dialkylpolysiloxane or mixtures thereof.

The trialkylsilyl terminated dialkylpolysiloxanes used in the formation of silicone emulsion of the present invention are preferably those of the formula

R′₃SiO (R′₂SiO)ₚSiR′₃ (III),

wherein, R′ is a monovalent hydrocarbon radical having from 1 to 18 carbon atoms and

p is a number of from 500 to 2000, preferably of from 1000 to 2000.

The trialkylsilyl terminated (or end-blocked) dialkylpolysiloxanes have a viscosity of from 5 to 100 mPa·s at 25° C., preferably a viscosity of from 40 mPa·s to 70 mPa·s at 25° C. The trialkylsilyl endblocked polydimethylsiloxanes may have approximately 100 ppm by weight of polydimethylsiloxanes having hydroxyl end groups. The trialkylsilyl terminated dialkylpolysiloxane according to the invention are preferably linear but may contain additionally to the R′₂SiO_{2/2} units (D-units) in formula (I) RSiO_{3/2} units (T-units) or SiO_{4/2} units (Q-units), wherein R′ is a monovalent hydrocarbon radical having from 1 to 18 carbon atoms.

Examples of hydrocarbons R′ are alkyl radicals, such as the methyl, ethyl, n-propyl, isopropyl, 1-n-butyl, 2-n-butyl, isobutyl, tert-butyl, n-pentyl, isopentyl, neopentyl and tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radicals, decyl radicals, such as the n-decyl radical, dodecyl radicals, such as the n-dodecyl radical, and octadecyl radicals, such as the n-octadecyl radical; alkenyl radicals such as the vinyl and ally radical; cycloalkyl radicals, such as the cyclopentyl, cyclohexyl, cycloheptyl and methylcyclohexyl radicals; aryl radicals, such as the phenyl, naphthyl, anthryl and phenanthryl radical; alkaryl radicals, such as the o-, m- and p-tolyl radicals, xylyl radicals and ethylphenyl radicals; and aralkyl radicals such as the benzyl radical and the and the phenylethyl radical. Most preferred is the methyl radical.

Most useful emulsifiers of this category are polyoxyalkylene alkyl ether, polyoxyalkylene alkylphenyl ethers and polyoxyalkylene sorbitan esters. Some useful emulsifiers having HLB value in between 10-16 are polyethylene glycol octyl ether; polyethylene glycol lauryl ether; polyethylene glycol tridecyl ether; polyethylene glycol cetyl ether; polyethylene glycol stearyl ether; polyethylene glycol nonylphenyl ether; polyethylene glycol dodecylphenyl ether; polyethylene glycol cetylphenyl ether; polyethylene glycol stearylphenyl ether; polyethylene glycol sorbitan mono stearate and polyethylene glycol sorbitan mono oleate. Non-ionic emulsifiers or its mixture having HLB value in-between 10-16 has a great importance in the present invention to make process simpler. In another embodiment the emulsifier is most preferably a mixture of nonionic emulsifiers.

The emulsifiers of the invention are PEG-100 STEARATE and TRIDECETH-10. PEG 100 Stearate is of chemical formula CH₃(CH₂)₁₆CO(OCH₂CH₂)₁₀₀OH (HLB value 18.8), Trideceth-10 is of chemical formula CH₃(CH₂)₁₂(OCH₂CH₂)₁₀—OH (HLB value 13.25).

In another embodiment a cationic emulsifiers may be selected as an emulsifier. Examples of the cationic emulsifiers are selected from tetra alkyl ammonium halides, tetra aryl ammonium halides, tetra alkyl aryl ammonium halides, also including salt, quaternary ammonium compound including salt, polyquaternium compound having INCI name polyquaternium 1 to 75 in use together with said non-ionic emulsifiers. The most preferable cationic emulsifier is cetyltrimethyl ammonium chloride (CTAC). Such emulsifier is obtained as Arquad from AkzoNobel, Cetyltrimethylammonium chloride solution from Sigma-Aldrich.

In one of the embodiments, the anionic surfactant is selected from the group consisting of alkylaryl sulfonic acid; alkylaryl polyoxyethylene sulphonic acid; alkyl sulfonic acid, alkyl polyoxyethylene sulfonic acid, and mixtures thereof. The most preferable anionic surfactants used in the present inventions are octylbenzene sulfonic acid; dodecylbenzene sulfonic acid; cetylbenzene sulfonic acid; alpha-octyl sulfonic acid; alpha-dodecyl sulfonic acid; alpha-cetyl sulfonic acid; polyoxyethylene octylbenzene sulfonic acid; polyoxyethylene dodecylbenzene sulfonic acid; polyoxyethylene cetylbenzene sulfonic acid; polyoxyethylene octyl sulfonic acid; polyoxyethylene dodecyl sulfonic acid; and polyoxyethylene cetyl sulfonic acid. Generally, 1 to 15% anionic surfactant is used in the present emulsion process.

In one of the embodiments, the non-ionic emulsifier is selected from alkyl ether of polyalkylene glycol and alkyl esters of polyalkylene glycol or mixtures thereof.

In another embodiment, the emulsion the mixture of emulsifier further comprises optionally one or more cationic or anionic emulsifier or mixtures thereof.

HLB values are typically referred to the values at room temperature. As temperature varies, the HLB value of a surfactant may also vary. Calculation of HLB value of non-ionic surfactants is calculated according to the equations provided according to the terms of the HLB system of emulsifier classification introduced by Griffin, W. C. ibid 5 249 (1954).

For ionic surfactants, the HLB value of individual surfactant molecules can be calculated applying the Davies formula as described in Davies JT (1957), “A quantitative kinetic theory of emulsion type, I. Physical chemistry of the emulsifying agent”, Gas/Liquid and Liquid/Liquid Interface (Proceedings of the International Congress of Surface Activity): 426-38. According to the formula the HLB is derived by summing the hydrophilic/hydrophobic contributions afforded by the structural components of the surfactant.

HLB=(hydrophilic group numbers)−n (group number per CH2 group)+7

For example, Tetradecyl trimethyl ammonium chloride has the following structure:

CH₃—(CH₂)₁₃N⁺—(CH₃)₃Cl⁻

Group contribution of the hydrophobic groups: —CH₂/—CH₃−0.475

Group contribution of the hydrophilic group: N⁺—(CH₃)₃ Cl⁻22.0

HLB=22−(14×0.475)+7=22.4

Approximate H LB values for some cationic emulsifier are given in Table IV, in

Cationic emulsifiers in cosmetics, K. M. GODFREY, J. Soc. Cosmetic Chemists 17 17-27 (1966).

The composition according to the invention is an oil-in-water emulsion. In an emulsion, one liquid (the dispersed phase) is dispersed in the other (the continuous phase). According to the inventive oil-in-water emulsion the silicone mixture (the dispersed phase) is dispersed in the continuous water phase. The emulsion may be a micro emulsion or a macro emulsion. The composition may also be in the form of water-in-oil or multiple emulsions, a solution, a suspension, dispersion, an aerosol, microcapsules or micro particles. Preferentially, the composition according to the invention may be an oil-in-water or water-in-oil emulsion. More preferentially, the composition according to the invention is an oil-in-water emulsion.

The stability of the emulsion is determined by the circulation of the emulsion at 45° C. for 3 months and the stability will be determined by no change in property of the emulsion. If the property changes or the oil and water phase separates at the above condition, the emulsion is said to be unstable.

The viscosity of the fluids, its mixture and the emulsion prepared by the fluid is measured at 25° C. by Anton Paar Rheometer; model MCR101, geometry single gap cylinder: CC27 spindle and shear rate 1 s⁻¹ for 2 minutes at 25° C. is used for viscosity between 1000 to 15,000 mPa·s at 25° C. Three measurements are made for each sample and the viscosity value is taken at 60 secs. A MCR Rheometer Series product works as per USP (US Pharmacopeial Convention) 912—Rotational Rheometer methods.

Emulsion particle size is measured by using a device ZetaSizer from Malvern, UK, model Nano-ZS which is based on the Photon Correlation Spectroscopy (PCS) method. The D₅₀ value of particle size (average hydrodynamic particle diameter) is measured, wherein the evaluating algorithm is “cumulants analysis”. Take 0.5 g of the emulsion sample in a 250 ml beaker, 100 ml of DM water is poured into it and then mixed properly to get the sample test solution. The sample test solution is poured in the cuvette cell and is put into the slot of the instrument to measure the particle size of the emulsion. D₅₀ is defined as the value of the particle diameter at 50% in the cumulative distribution. For example, if D₅₀=170 nm, then 50% of the particles in the sample are larger than 170 nm, and 50% smaller than 170 nm or about 50% by volume of all droplets in said emulsion is 170 nm.

In one embodiment, the particle size of the micro emulsion is less than 500 nanometers and preferably less than 350 nanometers. In another embodiment, the particle size of the macro emulsion is less than 5 micrometers and preferably less than 3 micrometers.

The oil-in-water emulsion of the invention is preferably used in consumer product including a personal care product such as skin or hair care composition. Preferably, the oil-in-water emulsion of the invention is used in hair care compositions, preferably in amounts of from 1 to 15 percent by weight based on the hair care composition.

The method of preparing the emulsion further comprises adding a biocide. Biocide was added for preserving the emulsion against microbial contamination. The biocide is added at the level of for preserving emulsion against microbial contamination and obtaining the said emulsion. The quantity of the biocide depends on the type of biocide and as recommended by the manufacturer.

Further constituents of the hair care compositions are, for example, surfactants, fatty alcohols, rheology modifiers, pearlizers, organic acids, fragrances, preservatives, vitamins, sunscreens, salts, dyes, and further components of hair care compositions known to those skilled in the art.

“Consumer products” as used herein is defined as the products made for and used by consumers for personal and home care. Personal cares are primarily the area of hair care and skin care. The hair care compositions may be e.g. shampoos, rinses, creams, sprays, shampoo, and for skin care product such as soap, liquid bath. These care compositions improve both the dry and the wet combability, and also the feel to the touch in the wet and dry hair. Application can take place e.g. during washing, after washing, as pre- or after-treatment during bleaching or during coloring with direct or oxidation dyes, and during the permanent shaping of hair (e.g. permanent wave). The invention further provides hair care compositions comprising emulsions that impart a better shine property along with other desirable properties. Other desirable properties include but not limited to alignment of hair, better feel, and consumer feel faster clean feel due to faster removal of shampoo residues from hair during rinsing. The composition may further be used in other applications like fiber (natural or artificial) alignment and thus improving shine and further feel improvement.

The details of the invention, its nature and objects are explained hereunder in greater detail in relation to the following non-limiting examples.

### Synthesis Examples

### Example 1: Synthesis of Silicone Fluid Comprising a Glycoside Unit

1000 g of caprylyl dimethicone ethoxy glucoside (SPG128 VP available from Wacker having INCI name CYCLOPENTASILOXANE, CAPRYLYL DIMETHICONE ETHOXY GLUCOSIDE), 4000g of polydimethyl siloxane (PDM) fluid and 100 g of trimethyl terminated dimethyl siloxane (AK-10 fluid available from Wacker) of viscosity 50 mPa·s at 25° C. is loaded in the reactor. The temperature is set to 150° C. under N₂ (5 LPH). When temperature reaches 150° C. add 5 g of KOH-H₂O solution catalyst and continue stirring under N₂ (10 LPH) for 1.5 hrs. After 1.5 h, again add 5 g KOH—H₂O catalyst at 150° C. and continue stirring under N₂ (30 LPH) for 1.5 hrs; reflux starts after this catalyst addition. After 1.5 h add 3 gm KOH—H₂O catalyst (13 g of KOH to form 50% solution of KOH) at 150° C. and continue stirring under N₂ (40LPH). Viscosity of the polymer is checked at 150° C. time to time and when viscosity reaches 750-850 mPa·s (at 150° C.), neutralize the mixture with 13.7 g acid at 150° C./N₂ (SLPH) with stirring for 40 min. Wt. loss: 17.7% at 200° C. 15 min 0.5 g; Distillate=70 g; H₂O=9.1 g. After that all volatiles are stripped off under full vacuum at 150° C. for 2 h. Material is cooled down and collected after filtration through 400 Mesh cloth.

Appearance=Clear deep red oil of viscosity=9,700 cps at 25° C. Weight loss=1.34% at 200° C. 15 min 0.5 g. Yield=4055 g (79%) Distillate=778 g.

### Example 2 (a): Preparation of Oil-in-Water Emulsion of Silicone Fluid of Example 1 with Non-Ionic Emulsifier

Emulsion is made at 20° C. by homogenization. First a paste is made with 48 grams of Trideceth 10, and then 90 grams of PEG-100 STEARATE (3.36%) and 120 grams of water (4.48%) is added by heating at 50° C. Then the paste is cooled and homogenized with1205 grams of silicone fluid and 34 grams of 2-phenoxyethanol. Finally the emulsion is diluted with 1180 grams of water to 45% active to get an oil-in-water emulsion of 200 nm D₅₀ particle size.

### Example 2 (b): Preparation of Oil-in-Water Emulsion of Silicone Fluid of Example 1 with Non-Ionic Emulsifier and Cationic Emulsifier

Emulsion is made at 20° C. by homogenization. First a paste is made with 17.6 grams of Trideceth 10 (1.77%), and then 34 grams of PEG-100 STEARATE (3.42%) and 48.2 grams of water is added by heating at 50° C. Then the paste is cooled and homogenized with 450 grams of silicone fluid (45.20%), 2 g of guarhydroxypropyltrimmonium chloride (0.2%) and 10 grams of 2-phenoxyethanol (1%). Finally the emulsion is diluted with 433.8 grams of water (48.41%) active to get an oil-in-water emulsion of 255 nm D₅₀ particle size.

### Example 2 (c): Preparation of Oil-in-Water Emulsion of Silicone Fluid of Example 1 with Non-Ionic Emulsifier and Anionic Emulsifier

Emulsion is made at 20° C. by homogenization. First a paste is made with 17.6 grams of Trideceth-10 (1.77%), and then 34 grams of PEG-100 STEARATE (3.42%) and 46.4 grams of water is added by heating at 50° C. Then the paste is cooled and homogenized with 450 grams of silicone fluid (45.20%), 20 g of TEA-Dodecylbenzenesulphonate (2.01%) and 10 grams of 2-phenoxyethanol (1%). Finally the emulsion is diluted with 417.6 grams of water (46.61%) active to get an oil-in-water emulsion of 292 nm D₅₀ particle size.

### Comparative Example 1: Synthesis of Low Viscosity Silicone Fluid Comprising a Glycoside Unit

Load 1000 g of caprylyl dimethicone ethoxy glucoside (SPG128 VP available from Wacker having INCI name CYCLOPENTASILOXANE, CAPRYLYL DIMETHICONE ETHOXY GLUCOSIDE), 4000 g of polydimethyl siloxane (PDM) fluid and 200 g of trimethyl terminated dimethyl siloxane (AK-10 fluid available from Wacker) of viscosity 50 mPa·s at 25° C. in the reactor. The temperature is set to 150° C. under N₂ (5 LPH). When temp reaches 150° C. add 4 g of KOH—H₂O solution catalyst and continue stirring under N₂ (10 LPH) for 1.5 hrs. After 1.5 h, again add 5g KOH—H₂O catalyst at 150° C. and continue stirring under N₂ (30 LPH) for 1.5 hrs; reflux starts after this catalyst addition. After 1.5 h add 3 gm KOH—H₂O catalyst (13 g of KOH to form 50% solution of KOH) at 150° C. and continue stirring under N₂ (40LPH). Viscosity of the polymer is checked at 150° C. time to time and when viscosity reaches 500-600 mPa·s (at 150° C.), neutralize the mixture with 13.7 g acid at 150° C./N₂ (SLPH) with stirring for 40min. Wt. loss: 14.6% at 200° C. 15min 0.5 g; Distillate=177 g; H₂O=10.3 g. After that all volatiles are stripped off under full vacuum at 150° C. for 2 h. Material is cooled down and collected after filtration through 400 Mesh cloth to obtain the low viscosity silicone fluid. Appearance=Clear deep red oil of viscosity=4200 mPa·s at 25° C. Weight loss=1.29% at 200° C. 15 min 0.5 g. Yield=4180 g (80%) Distillate=697 g.

### Comparative Example 2: Preparation of Oil-in-Water Emulsion of Silicone Fluid of Comparative Example 1

Emulsion is made at 20° C. by homogenization. First a paste is made with 17.6 grams (1.77%) of Trideceth-10, then 34 grams of PEG-100 STEARATE (3.42%) and 45 g of water (4.52%) is added by heating at 50° C. Then the paste is cooled and homogenized with 1205 grams of the low viscosity silicone fluid of Comparative Example 1 and 10 grams of 2-phenoxyethanol. Finally the emulsion is diluted with 429 grams of water to 43% active to get an oil-in-water emulsion of 300 nm D₅₀ particle size at a pH of 6.87.

### Comparative Example 3: Emulsion of 20% SPG128 VP in Cyclopentasiloxane (D5) Solution

Emulsion is made at 20° C. by homogenization. First a paste is made with Trideceth 5=1%, Then PEG-100 STEARATE=5.32%, ceteareth-20=1% and water=4.52% by heating at 50° C. Then the paste is cooled and homogenized with 20% SPG128 VP in cyclopentasiloxane (D5) solution and phenoxyethanol. Then rest Trideceth-5=4.32% is added and finally the emulsion is diluted with water to 32% active. The D₅₀ emulsion particle size is 150-230 nm at a pH of 6.87. The emulsion is not stable and becomes unstable overtime.

### Comparative Example 4: Emulsion with Alkyl Polyglycoside (Alkylpolyglycoside C8-10) Emulsifier and Polydimethylsiloxane Fluid

300 g of alkylpolyglycoside C₈-C₁₀ (CAS No. 68515-73-1) (30%), 300 g of polydimethyl siloxane (PDM) fluid of viscosity of 10000 mPa·s at 25° C. (30%) and 360 g of water (36%) is added together and 40 g of melted glyceryl monostearate (4%) was added and homogenized.

### Example 3: Preparation of Shampoo Base

The ingredients of list 1 as described in Table 1 is taken in a reactor and stirred at ambient temperature i.e. 25° C. for 1 hour and then gradually the temperature is raised to 45 to 50 ° C.

The ingredients of list 2 are added and stirred for 2 hours and then cooled to room temperature.

All the ingredients of list 3 are then added and stirred for 2 hours. The standard shampoo (standard shampoo) composition is prepared separately by adding silicone emulsion with INCI Dimethiconol (and) TEA-dodecylbenzenesulfonate, instead of silicone fluid emulsion of the invention for performing the evaluation tests.

TABLE 1
Shampoo base composition

| List of | | Quantity |
|---|---|---|
| Ingredients | Ingredients | (wt %) |
| List 1 | Demineralised water | 50 |
| | Jaguar C17S (Guar Hydroxylpropyl | 0.08 |
| | trimonium chloride) | |
| | Jaguar C14S (Guar Hydroxylpropyl | 0.15 |
| | trimonium chloride) | |
| | Lysine Hydrochloride | 0.01 |
| | Carbomer/Carbopol 980 | 0.4 |
| | Citric acid | 0.1 |
| List 2 | Sodium Lauryl ether sulphate 2EO | 18.26 |
| | (70% active) | |
| List 3 | Cocoamidopropyl betaine | 4.4 |
| | (37% active) | |
| | Silicone fluid emulsion of example | 6.6 |
| | 2 (a) of the invention (shampoo 1a), | |
| | example 2 (b) of the invention | |
| | (shampoo 1b), example 2 (c) of the | |
| | invention (shampoo 1c) and separately | |
| | Comparative Example 2 (Shampoo 2), | |
| | Comparative Example 3 (Shampoo 3) | |
| | and Comparative Example 4 | |
| | (Shampoo 4) | |
| | Glydant (DMDM Hydantoin) | 0.10 |
| | Kathon CG | 0.12 |
| | Sodium Chloride | 1.80 |
| | Sodium Hydroxide | 0.20 |
| | Disodium EDTA | 0.20 |
| | Demineralized water | q.S to 100 |

### Evaluation Tests with Shampoo Base as Prepared in Example 3

### Measurement of Friction and Combing Force

Take the hair tresses to be tested and immerse in Pet-ether (60-80° C. boiling range) for 1 hr. After 1 hr dry them in open air. Then wash with sodium lauryl ether sulfate (SLES). Dry them again in open air.

The test methods that are performed to determine the conditioning property are by measure the friction and combing force after applying silicone fluid emulsion of the invention in shampoo on hair tresses by using Texture Analyzer from Stable Micro Systems Machine. The metallic comb is attached horizontally and tightened with the screws. Now fix the hair tress within the clip of upper zig. The comb height is calibrated. The speed of the comb is 5 mm/s and the test is run for 10 times. The result is obtained in miliNewton. The test results are depicted in Table 2 shows the lower dry combing force and frictional force of shampoo base with silicone fluid emulsion of example 2 (a), (b) and (c) of the invention, which shows better silicone deposition properties, and hence improved conditioning properties.

### Silicone Deposition Test

The amount of deposition of silicone on the hair sample after initial treatment with the hair care composition was determined using X-ray diffraction (XRD). The three hair samples for each hair care composition were blended and analyzed for % relative silicon concentration on the hair surface. XRD statistics were generated from 5 measurements discarding the highest and lowest result.

Each hair sample is washed by 0.5 mL of a detergent solution having 12% sodium lauryl ether sulfate (SLES)). The detergent solution was manually distributed throughout the hair sample along the length of the hair sample from top to bottom by repeated downward motions for 30 seconds. The hair sample was then placed under running warm water, at a temperature between 100 to 110° F., and rinsed for 30 seconds. The hair sample was subsequently dried using a blow-dryer at 25° C. and at 40-50 relative humidity (RH). The washing and drying steps were repeated two times. The amounts of deposition of silicone on the hair sample after the second washes were determined using XRD. The ideal silicone deposition level is determined to be in the level of 750 ppm to 850 ppm.

### Measurement of Shine

The initial shine evaluation is done visually with 10 Participants so as to have a spread of respondents feel. Each participant has provided feedback according to better than the other when treated with shampoo 1 to 4 and standard shampoo as depicted in Table 1. The shine evaluation is mentioned below from High to low shine evaluation as follows:

Shampoo 1(a)˜Shampoo 1(b)˜Shampoo 1(c)>Shampoo 2˜Standard shampoo>Shampoo 3˜Shampoo 4

TABLE 2
DRY COMBING FORCE and FRICTION TEST:

| | | Dry | | Silicone |
|---|---|---|---|---|
| | | Combing | Dry | Deposition |
| | | Force, | friction, | Value |
| | Sample | mN | mN | (ppm) |
| Shampoo | Shampoo base with | 3675 | 180 | 800 |
| 1(a). | Silicone fluid emulsion | | | |
| | of example 2(a) of the | | | |
| | invention | | | |
| Shampoo | Shampoo base with | 3225 | 172 | 805 |
| 1(b). | Silicone fluid emulsion | | | |
| | of example 2(b) of the | | | |
| | invention | | | |
| Shampoo | Shampoo base with | 3690 | 182 | 789 |
| 1(c). | Silicone fluid emulsion | | | |
| | of example 2 (c) of the | | | |
| | invention | | | |
| Shampoo | Shampoo base with | 3985 | 193 | 690 |
| 2. | silicone fluid emulsion | | | |
| | of comparative | | | |
| | example 2 | | | |
| Shampoo | Shampoo base with | 7961 | 298 | 570 |
| 3. | emulsion of | | | |
| | Comparative Example | | | |
| | 3 | | | |
| Standard | Standard shampoo | 4012 | 213 | 710 |
| shampoo | | | | |
| Shampoo | Shampoo base with | 5930 | 329 | 615 |
| 4. | emulsion of | | | |
| | Comparative Example | | | |
| | 4 | | | |

### Salon Test

Again to confirm the finding a SALON TEST was performed for the Silicone fluid emulsion of example 2 of the invention (shampoo 1) with respect to the standard shampoo as depicted in Table 3 below.

A salon test is performed by performing half head salon—shampoo only where the target group is female of Age group: 18-35 yrs. Salon was performed in three different salons in two different locations with 14 Participants in each salon (totaling to 84 participants) so as to have a good spread of respondents feel.

Each participant has provided feedback according to better than the other when treated with prototype shampoo and standard shampoo. The participants uniformly followed the following schedule before the salon trial:

Regular & current user of Market Shampoo

Participants do hair washing at least thrice a week

The feedback of the Salon test is captured in Table 3.

TABLE 3
Salon Feed Back Report

| | | | Prototype | | |
|---|---|---|---|---|---|
| | | | Shampoo | | |
| | | | shampoo | Standard | |
| | | | with silicone | Shampoo | |
| | | | fluid emulsion | (No. of | |
| | | | (No. of | people | |
| | | | people who | who gave | |
| | | Sample | gave feedback | feedback | Confidence |
| | | Size | in favor) | in favor) | % |
| APPLICATION | speed of time to lather | N = 84 | 42 | 42 | |
| | amount of lather | N = 84 | 45 | 39 | |
| | foam stability | N = 84 | 48 | 36 | |
| RINSING | less time to rinse | N = 84 | 64 | 20 | 99.98 |
| | slippery feel of hair | N = 84 | 48 | 36 | |
| WET STAGE | ease of comb | N = 84 | 45 | 39 | |
| | most slippery while | N = 84 | 45 | 39 | |
| | combing | | | | |
| DRY STAGE | ease of comb | N = 84 | 51 | 33 | 99.98 |
| | volume/fullness of hair | N = 84 | 45 | 39 | |
| | shine of hair | N = 84 | 64 | 20 | |
| | slippery feel of hair | N = 84 | 51 | 33 | |
| | smoothness of hair | N = 84 | 51 | 33 | |
| | feel of hair after dryness | N = 84 | 48 | 36 | |
| | less heaviness in hair | N = 84 | 45 | 39 | |

The following evaluation test suggests that the confidence percentage of shine and time needed to rinse is 99.98%. Thus showing improved shine and improved rinsing benefits along with reduced dry combing force and increased deposition thus showing the improved conditioning properties.

### Example 4: Preparation of Oil-in-Water Big Blob Emulsion

Emulsion is made at 20° C. by homogenization. First a paste is made with 1.6 grams of Trideceth-10. Then adding 32 grams of PEG-100 stearate and 150 grams of water by heating at 50° C. The paste is then cooled and homogenized with 384 grams of silicone fluid of example 1 and 8 grams of 2- phenoxyethanol. Finally the 60 g of water is added to obtain a stable oil-in-water-emulsion of 65.6% active, having pH 7 with D₅₀ emulsion particle size of 2.5 microns.

### Example 5: Preparation of Beauty Conditioner Base

All ingredients of List 1 of Table 3 are mixed one by one at 55° C., stirring each component for 20 minutes each and ensure the phase is uniform. Then mixing ingredients of List 2 of Table 3 at 30° C. for 10 minutes after cooling. Then mixing ingredients of List 3 of Table 3 at 30° C. for 15 minutes. For preparing standard conditioner composition the oil-in-water big blob emulsion of Example 4 of current invention is replaced by same amount of a silicone oil-in-water emulsion composition of INCI name dimethicone/amino silicone emulsion which forms a standard conditioner, all the ingredients of Table 3 of the beauty conditioner base composition remains same.

TABLE 3
Beauty conditioner bas composition

| Ser. | | Quantity |
|---|---|---|
| No. | Ingredients | (wt. %) |
| List 1 | Demineralized water | 87.51 |
| | Behentrimonium chloride | 1.63 |
| | Glycerine | 1.0 |
| | Stearamidopropyl Dimethylamine | 0.38 |
| | Cetearyl Alcohol (Ginol 16-18 TA) | 4 |
| List 2 | Methyl Paraben | 0.2 |
| | Lactic Acid/Purac PH 90(90%) | 0.12 |
| | MIT/Neolone 950(9.5%) | 0.27 |
| | Glydent 40-700 (DMDMHN)-55% | 0.27 |
| List 3 | oil-in-water big blob emulsion of | 4.62 |
| | Example 4 of current invention | |

### Evaluation Test

Evaluation of silicone conditioner on hair tresses manually The hair tresses to be tested and immerse in Pet-ether (60-80° C. boiling range) for 2 hrs. After 2 hr, decant the washed solvent and again add fresh pet ether, immerse again for 2 hrs more. Dry them in open air. Then wash with detergent (use 2 gm detergent per 100 gm hair).Dry them again in open air. Take half portion of the conditioner (usually 1 gm of the total two gms) to be applied and apply it along the whole length of the tress for 30 s. Keep it untouched for 30 s. Then wash it off properly. Again apply the rest portion of the conditioner composition similar way for 60 s and keep it un-touched for 60 s. Then finally washes it off with water properly.

Try to feel the wet softness and rated as number of participants who gave feedback in favor. Count the number of strokes for detangling completely in wet condition, rated as number of participants who gave feedback in favor. Allow it to dry in open air.

Try to feel the dry softness and rate it as rated as number of participants who gave feedback in favor. Count the number of strokes for detangling completely in dry condition.

### Study of Shine of Hair

To study shine property of hair visually by means of GTI Minimatcher from HOSTECH. The treated hair tresses and place on the viewing surface of the booth. The hair tresses are observed under three lighting conditions—store light, incandescent light and daylight inbuilt in the machine itself.

14 people panel were formed for evaluation. The final ranking is taken as average of the 14 individual rankings.

TABLE 4
Evaluation of silicone conditioner and shine properties

| | | Conditioner with | |
|---|---|---|---|
| | | silicone emulsion | Standard |
| | | of example 4 | Conditioner |
| | | (No. of | (No. of |
| | | people who | people who |
| | | gave feedback | gave feedback |
| | | in favor) | in favor) |
| WET | Wet softness | 10 | 2 |
| STAGE | the number of strokes for | 9 | 5 |
| | detangling (Numbers) | | |
| DRY | the number of strokes for | 11 | 3 |
| STAGE | detangling (Numbers) | | |
| | shine of hair in daylight | 10 | 4 |
| | shine of hair in store light | 10 | 4 |
| | shine of hair in | 8 | 6 |
| | incandescent light | | |
| | feel of hair after dryness | 10 | 4 |

Thus we see from the table 4 that the shine and conditioning benefits improve for the conditioner with silicone fluid emulsion.

1. An oil-in-water emulsion comprising:
a) a silicone fluid of formula (I)
R⁵₃SiO(R¹₂SiO)ₘ(R¹R²SiO)ₙ(YR¹SiO)ₚ—SiR⁵₃ (I)
where, Y is a radical of formula Z(R³O)_{f}R⁴—,
n and p is from 0 to 10000 and m is from 200 to 10000,
R¹ is selected from a C₁ to C₆ alkyl group, R² is a C₁ to C₁₈ alkyl group,
R³ is a C₁ to C₆ alkylene radical,
R⁴ is a C₁ to C₆ alkylene radical,
R⁵ is selected from a C₁ to C₆ alkyl group, C₁ to C₆ alkoxy group,
hydroxyl group,
Z is a glycoside radical of from 1 to 100 monosaccharide units or its derivatives;
b) a mixture of emulsifier comprising one or more non-ionic emulsifier; and
c) water,
wherein, the silicone fluid is of viscosity of at least 4500 mPa·s at 25° C. and the non-ionic emulsifier is selected from alkyl ether of polyalkylene glycol and alkyl esters of polyalkylene glycol or mixtures thereof.

2. The emulsion of claim 1, having a D₅₀ particle size less than 3 micrometers.

3. The emulsion of claim 1, wherein the R² is a C₂ to C₁₈ alkyl group.

4. The emulsion of claim 1, wherein Z is a glycoside radical of from 1 to 10 monosaccharide units or its derivatives.

5. The emulsion of claim 1, wherein the mixture of emulsifier further comprises one or more cationic or anionic emulsifier or mixtures thereof.

6. The silicone fluid of claim 1, wherein the silicone fluid is having less than 1000 ppm by weight of hydroxyl terminated dialkylpolysiloxane or methoxy terminated dialkylpolysiloxane or mixtures thereof.

7. A personal care composition comprising:
an emulsion comprising:
a) a silicone fluid of formula I of viscosity of at least 4500 mPa·s at 25° C.;
b) a mixture of non-ionic emulsifier; and
c) water,
wherein, the silicone fluid is of viscosity of at least 4500 mPa·s at 25° C. and the non-ionic emulsifier is selected from alkyl ether of polyalkylene glycol and alkyl esters of polyalkylene glycol or mixtures thereof.

8. The personal care composition of claim 7, which is in the form of a conditioner or a shampoo, soap, liquid bath, skin care product.

9. A method of preparing a silicone oil-in-water emulsion having a D₅₀ particle size less than 3 micrometers comprising:
rearranging caprylyl dimethicone ethoxy glucoside of viscosity from 40 to 100 mPa·s at 25 ° C. with polydimethylsiloxane of viscosity from 40 to 100 mPa·s at 25 ° C. and trialkyl terminated polydialkyl siloxane of viscosity from 5 to 50 mPa·s at 25 ° C. at a temperature of 100 to 175 ° C. to obtain a silicone fluid of viscosity of at least 4500 mPa·s at 25° C.;
emulsifying the silicone fluid with a mixture of emulsifier comprising one or more non-ionic emulsifier and water to obtain the silicone oil-in water emulsion.

10. The process of claim 9, wherein the mixture of emulsifier further comprises one or more cationic or anionic emulsifier or mixtures thereof.

11. A personal care composition comprising:
a silicone fluid of formula (I)
R⁵₃SiO(R¹₂SiO)ₙ(R¹R²SiO)ₘ(YR¹SiO)ₚ—SiR⁵₃ (I)
where, Y is a radical of formula Z(R³O)_{f}R⁴—,
n and p are numbers from 0 to 10000 and m is a number from 200 to 10000,
R¹ is selected from a C to C6 alkyl group, R2 is a C to C18 alkyl group,
R³ is a C₁ to C6 alkylene radical,
R⁴ is a C₁ to C6 alkylene radical,
R⁵ is selected from a C₁ to C₆ alkyl group, C₁ to C₆ alkoxy group, hydroxyl group,
Z is a glycoside radical of from 1 to 100 monosaccharide units or its derivatives,
wherein, the silicone fluid is of viscosity of at least 4500 mPa·s at 25° C. and the non-ionic emulsifier is selected from alkyl ether of polyalkylene glycol and alkyl esters of polyalkylene glycol or mixtures thereof.

12. The personal care composition of claim 11, further comprises a cosmetic solvent.
